(19) United States
(12) Patent Application Publication
(10) Pub. No.: US 2001/0037065 A1
(43) Pub. Date: Nov. 1, 2001

(54) Introducer sheath

(75) Inventors: Matthew M. Graf, Bloomington, IN (US); David A. Drewes JR., Bloomington, IN (US); Scott E. Eels, Bloomington, IN (US)

Correspondence Address: COOK GROUP PATENT OFFICE, P.O. BOX 2269, BLOOMINGTON, IN, 47402

(73) Assignee: Cook Incorporated, Bloomington, IN (US)

(21) Appl. No.: 09/810,377

(22) Filed: Mar. 16, 2001

Related U.S. Application Data
(60) Provisional application No. 60/191,058, filed on Mar. 21, 2000.

(51) Int. Cl. A61B 5/00

(52) U.S. Cl. 600/452; 600/585

(57) ABSTRACT

An introducer sheath (30) having a short distal tip section (34,40) that is highly radiopaque. The distal tip section may be of FEP with 20% to 75% by weight tungsten particulate filler, and may be initially a separate member (40) and bonded to the sheath shaft distal end (32).

### RELATED APPLICATION INFORMATION

This application claims priority from U.S. Provisional Patent Application Ser. No. 60/191,058 filed Mar. 21, 2000.

### FIELD OF THE INVENTION

The present invention relates generally to the field of medical devices and more particularly to introducer sheaths.

### BACKGROUND OF THE INVENTION

An introducer sheath is utilized in the percutaneous placement of a guide wire or catheter into a blood vessel, and comprises a flexible tube that itself is introduced into the blood vessel over a dilator. Once in position, the dilator is removed from within the sheath and withdrawn from the patient, and the guide wire or catheter is inserted through the sheath into the patient. Such sheaths are of biocompatible polymeric material and preferably contain an amount of radiopaque material in the polymeric matrix, and include a short tapered distal tip portion. Sheaths should have sufficient radial rigidity to remain open or patent upon removal of the dilator, but be sufficiently flexible to permit manipulation without kinking, under conditions of normal use. Internal sheath diameters range from 4 French to 26 French (1.3 mm to 8.7 mm) to accommodate the outside diameters of dilators and catheters and wire guides to extend therethrough.

Introducer sheaths are known that include adjacent to the distal tip portion, a radiopaque marking distinct from the remainder of the sheath, to indicate through fluoroscopy the position of the distal tip portion of the sheath within the patient, to assure proper positioning. The sheath can be of fluorinated ethylene propylene (FEP) having about 5 to 40% by weight loading of barium filler. Introducer sheaths have been known that include an annular ring of radiopaque paint on the sheath adjacent to the distal tip. Also, such marking typically can be an annular band of platinum alloy, or tungsten or gold or the like that is secured within the outer surface of the sheath adjacent to the distal tip, as in the CHECK-FLO PERFORMER Introducer Sheath sold by Cook Incorporated, Bloomington, Ind. The metal band is spaced approximately one-quarter inch from the distal tip and imparts substantial rigidity to the somewhat flexible sheath, whereas it would be desirable for the sheath to flex sufficiently during positioning to temporarily assume an oval cross-section locally.

It has been known to provide catheters such as introducer catheters with elongate flexible soft distal tip portions to minimize vessel wall trauma. It has been known to provide such distal tip portions as initially separate members that are bonded to the distal end of the catheter tube, with the tip member having filler material therein for viewing by fluoroscopy. The catheter shaft may be of a multiple layer construction using different materials and may include a wire coil to maintain lumen patency. Catheter constructions utilizing initially separate distal tip members bonded to a shaft, are disclosed in U.S. Pat. Nos. 4,898,591; 5,045,072; 5,300,048; 5,584,821; and 5,769,830. However, such tip members are commonly made of copolymers that can be substantially loaded such as by tungsten, barium or bismuth, while the remainder of the catheter shaft contains substantially less radiopaque material adjacent to the distal tip portion.

It is desired to provide an introducer sheath in which the radiopaque marking is exactly at the distal tip rather than spaced slightly proximally from the tip, to best assure exact positioning by the surgeon.

### SUMMARY OF THE INVENTION

The foregoing problems are solved and a technical advance is achieved in an illustrative introducer sheath that includes a short distal tip section that is substantially more radiopaque than the radiopaque material of the remainder of the polymeric sheath shaft proximally from the distal tip. The distal tip may be a short initially separate ring of polymeric material affixed onto the distal end of the sheath shaft to define the distal tip section. The ring is made preferably of fluorinated ethylene propylene (FEP) containing a filler of tungsten or similar metal particles between about 20 to 75% by weight, while the sheath shaft is also of FEP with a substantially lower radiopaque filler content.

The present invention also is directed to a radiopaque composition of fluorinated ethylene propylene containing a loading of between about 20% to about 75% radiopaque filler, thereby being highly radiopaque, with the filler being tungsten, tantalum, platinum, gold, or lead or other metal.

### BRIEF DESCRIPTION OF THE DRAWINGS

An embodiment of the introducer sheath of the present invention will now be described by way of example with reference to the accompanying drawings.

FIG. 1 is an illustration of a Prior Art introducer sheath containing a metal radiopaque band proximate the distal tip;

FIG. 2 is an enlarged partial cross-section view of the distal tip region of an introducer sheath containing the present invention; and

FIG. 3 shows an initially separate tip member with filler.

### DETAILED DESCRIPTION

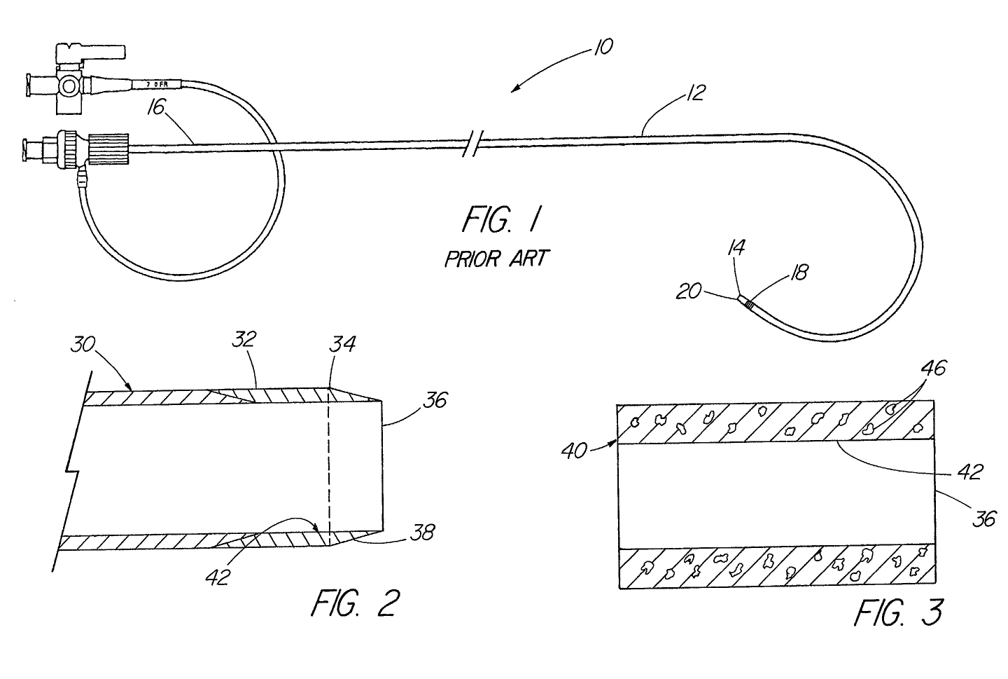

FIG. 1 illustrates an introducer sheath 10 of the prior art, having a shaft 12 having a distal tip 14 and a proximal end 16, and through which extends a lumen. Shaft 12 is polymeric, such as of fluorinated ethylene propylene and contains a radiopaque filler such as an 8 to 12% loading of barium sulphate. Adjacent to the distal end 14 is an annular band 18 of platinum alloy or gold that is highly radiopaque. Distal tip 14 has a tapered outer surface 20 to facilitate insertion into a patient, and metal band 18 embedded within the wall of sheath 10 and is spaced from distal tip 14 about one-quarter inch to assure against becoming dislodged during insertion and removal of the sheath from a patient. During use, a surgeon must estimate the exact location of distal tip 14 distally of the metal band 18, as discerned through fluoroscopy.

FIG. 2 illustrates the distal sheath portion containing the radiopaque distal tip section of the present invention. Sheath shaft 30 includes an end 32, with distal tip section 34 extending distally therefrom to a leading distal end 36 and having a tapered outer surface 38 thereat. Distal tip section 34 may be initially fabricated as a separate member 40 having a lumen 42 equal in diameter of lumen 44 of shaft 30, of a polymeric material that is at least similar enough to the polymeric material of the shaft to be easily and successfully bonded thereto. Such a member is easily extruded and cut to a short length, as shown in FIG. 3.

As an example, member 40 is extruded preferably from fluorinated ethylene propylene having dispersed therein a filler of tungsten particles 46 between about 20% and about 75% by weight, such as preferably about 50 to 55% by weight. The tungsten particles preferably range in size from about 0.5 microns to 25 microns, and more preferably are about 1.4 microns to about 1.8 microns in size. Other polymeric materials include nylon, polyethylene, polyurethane and polytetrafluoroethylene, and other radiopaque filler materials include tantalum, titanium, platinum, gold, silver, bismuth trioxide and lead and the like. It is unexpected that such high loading could be attained with FEP and still result in a stable extrudable composition that can be bonded at least to other FEP material. A loading of 20% tungsten results in a radiopacity that is roughly equivalent to that generated by a 40% loading of barium sulphate.

FEP sheaths have heretofore contained about 5 to 40% barium sulphate filler. Fluorinated ethylene propylene is not known to be fillable to over 40% with barium sulphate particles and still result in a stable extrudable composition. Generally, the particles of barium sulphate used in current introducer sheaths are between about 0.7 microns and 10 microns, preferably about 1 to 3 microns in size. It is believed that an irregular, nonspherical shape of metal particles, along with the high density of the metal, small particle size and narrow size distribution range, may permit such high loading levels in the present invention.

Member 40 can be cut to a length of for example one-quarter inch and be bonded onto an end of shaft 30 such as by adhesive or by thermal bonding, and thereafter be machined for finishing. One such thermal bonding method is disclosed in U.S. Pat. No. 5,017,259 for use with catheters. In accordance with U.S. Pat. No. 5,769,830, a thermal bond is attained by inserting a mandrel through the tubular shaft and the tip member and then inserted into a forming die to which radiofrequency energy is commonly applied for melting together the materials of the distal end portion of the sheath and the distal tip member.

What is claimed is:

1. An introducer sheath comprising:
a shaft extending from a proximal end portion to a distal end portion; and
a distal tip section at said distal end portion of said shaft,
said distal tip section comprising a polymeric material containing over 20% and up to about 75% by weight of radiopaque material, and
said shaft being distinctly less radiopaque than said distal tip section.

2. The introducer sheath according to claim 1, wherein said distal tip section contains between about 50% to 55% by weight of radiopaque material.

3. The introducer sheath according to claim 1, wherein said radiopaque material is selected from the group tungsten, titanium, tantalum, platinum, gold, silver, bismuth trioxide and lead.

4. The introducer sheath according to claim 1, wherein said radiopaque material is tungsten.

5. The introducer sheath according to claim 4, wherein said tungsten particles range in size from about 0.5 microns to about 25 microns.

6. The introducer sheath according to claim 4, wherein said tungsten particles range in size from about 1.4 microns to about 1.8 microns.

7. The introducer sheath according to claim 1, wherein said polymeric material of said distal tip section is selected from the group fluorinated ethylene propylene, nylon, polyethylene, polyurethane and polytetrafluoroethylene.

8. The introducer sheath according to claim 1, wherein said polymeric material of said distal tip section is fluorinated ethylene propylene.

9. The introducer sheath according to claim 8, wherein said polymeric material of said distal tip section contains radiopaque filler over 20% by weight of tungsten particles.

10. The introducer sheath according to claim 9, wherein said distal tip section contains between about 50% to 55% by weight of tungsten particles.

11. The introducer sheath according to claim 9, wherein said tungsten particles range in size from about 0.5 microns to about 25 microns.

12. The introducer sheath according to claim 9, wherein said tungsten particles range in size from about 1.4 microns to about 1.8 microns.

13. The introducer sheath according to claim 1, wherein said distal tip section was initially a separate member.

14. An introducer sheath comprising:
a shaft extending from a proximal end to a distal end; and
a distal tip section at said distal end of said shaft,
said distal tip section comprising a polymeric material containing radiopaque particles,
said shaft being distinctly less radiopaque than said distal tip section,
said distal tip section polymeric material is fluorinated ethylene propylene and contains between about 50% to 55% by weight of tungsten particles that range in size from about 1.4 microns to about 1.8 microns.
